(19) United States
(12) Patent Application Publication
(10) Pub. No.: US 2026/0077278 A1
(43) Pub. Date: Mar. 19, 2026

(54) AROMATIC EUTECTIC MIXTURES AND THEIR USE AS SOLVENTS, PARTICULARLY IN LIQUID-LIQUID SEPARATION

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE (CNRS), Paris (FR); Université Clermont Auvergne, Clermont Ferrand (FR)

(72) Inventors: Pauline Vergnory, Gif-sur-Yvette Cedex (FR); Guillaume Bertrand, Gif-sur-Yvette Cedex (FR); Jean-Michel Andanson, Clermont-Ferrand (FR)

(73) Assignees: COMMISSARIAT A L'ENERGIE ATOMIQUE ET AUX ENERGIES ALTERNATIVES, Paris (FR); CENTRE NATIONAL DE LA RECHERCHE SCIENTIFIQUE (CNRS), Paris (FR); Université Clermont Auvergne, Clermont Ferrand (FR)

(21) Appl. No.: 19/332,615

(22) Filed: Sep. 18, 2025

(30) Foreign Application Priority Data
Sep. 19, 2024 (FR) FR2409998

(51) Int. Cl. B01D 11/04; C07C 39/15; C07C 211/27; C07D 209/86; C07D 263/32

(52) U.S. Cl. CPC B01D 11/0492; C07C 39/15; C07C 211/27; C07D 209/86; C07D 263/32

(57) ABSTRACT

This disclosure concerns aromatic and polyaromatic eutectic mixtures and their uses as solvents, particularly in liquid-liquid separation. The mixtures may include compounds including di-tert-butyl-benzenes, biphenyls and terphenyls, polycyclic aromatic hydrocarbons selected from naphthalenes, acenaphthenes, anthracenes and pyrenes, fluorenes and carbazoles, 2,5-Diphenyloxazoles, compounds of formula (I), triphenylmethane, tribenzylamine, and 2,5-dibenzylphenol.

### CROSS-REFERENCE TO RELATED APPLICATION

This application claims priority to FR 2409998, filed Sep. 19, 2024, the disclosure of which is hereby expressly incorporated by reference herein in its entirety.

### BACKGROUND

This disclosure concerns aromatic and polyaromatic eutectic mixtures and their uses as solvents, particularly in liquid-liquid separation.

The extraction using an organic solvent is a method commonly used, particularly in chemical engineering and analytical chemistry, to extract, separate and purify one or more chemical compounds. In general, this involves taking one or more chemical species (or extractables) from a liquid or solid mixture, or from a suspension of a solid in a liquid.

Typically, an extraction medium is used to selectively extract one or more compounds from an initial mixture, on the basis of chemical or physical properties. The extraction medium is not or only slightly miscible with the main components of the initial mixture, and the compound to be extracted has more affinity with the extraction medium than with the main components of the initial mixture.

The extraction operation takes place in two parts: a first part in which the compound to be extracted is transferred between the initial mixture and the extraction medium; a second part in which the extraction medium is separated from the main mixture.

This means of extraction is generally an organic solvent.

In liquid-liquid extraction, one or more substances dissolved in a solvent (feed phase, usually aqueous) is extracted using another solvent (extraction solvent phase, usually organic) in which it is more soluble. The initial solvent and the extraction solvent must not be miscible. This technique is based on the differential solubility of the substances to be extracted in two immiscible liquid phases.

In solid-liquid extraction, the aim is to extract a substance present in a solid and transfer it into a liquid solvent. The maceration, infusion, and decoction techniques are solid-liquid extraction methods.

However, the organic solvents traditionally used in this context are generally highly flammable, which complicates their handling and storage and makes them risky. These organic solvents typically have low flash points, typically <100° C. This represents a major safety risk for these applications, which are likely to use large volumes of these solvents.

In addition, the organic solvents are often volatile and contribute to air pollution. Their poor management can lead to soil and water contamination. They also threaten the health of the operators concerned, including headaches, nausea and, in serious cases, neurological and/or carcinogenic/mutagenic effects.

The search for safer, less toxic and more environmentally-friendly solvents to minimize health and environmental risks while maintaining or even improving the efficiency of extraction processes is a priority. To overcome this problem of volatility, the researchers have created a number of polar and non-polar eutectic solvents. These eutectic solvents are mixtures of molecules with liquefaction points close to ambient temperature. However, these eutectic solvents have little affinity for many molecules, particularly those containing aromatic residues.

The aim of the disclosure is therefore to provide compositions that allow overcoming the above-mentioned disadvantages as a solvent, and in particular to provide for the first time a liquid aromatic eutectic solvent at room temperature.

One aim of the disclosure is to provide low-flammability compositions, in particular with flash points >100° C., which reduce the aforementioned risks, while at the same time being able to be used as an aromatic solvent.

Another aim of the disclosure is to provide such compositions, which are at least equivalent to, or even more effective than, the solvents traditionally used, for example toluene, particularly in liquid/liquid extraction.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Thus, according to a first aspect, the disclosure relates to a composition consisting of, consisting essentially of, or comprising at least two compounds chosen from:
Di-tert-butyl-benzenes;
Biphenyls and terphenyls, optionally substituted by two groups chosen independently from linear or branched C₁ to C₆ alkyls, the terphenyls being in particular ortho-terphenyls, meta-terphenyls or para-terphenyls, in particular para-terphenyls or ortho-terphenyls;
Polycyclic aromatic hydrocarbons selected from naphthalenes, acenaphthenes, anthracenes and pyrenes, which are optionally substituted by two groups independently selected from linear or branched C₁ to C₆ alkyls;
The fluorenes and carbazoles, which are optionally substituted by two or four groups independently selected from linear or branched C₁ to C₆ alkyls;
The 2,5-Diphenyloxazoles, optionally substituted by two groups independently selected from linear or branched C₁ to C₆ alkyls;
The compounds of formula (I):

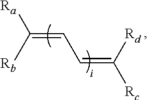

wherein:
i is equal to 0 or 1;
Rₐ, R_{b}, R_{c} and R_{d} are independently selected from H and phenyl, at least two of Rₐ, R_{b}, R_{c} and R_{d} being phenyl;
the compounds of formula (I) being optionally substituted, in particular on the phenyl groups, by two groups chosen independently from linear or branched C₁ to C₆ alkyls;
The triphenylmethane, optionally substituted, in particular on each phenyl group, by a group selected independently from linear or branched C₁ to C₆ alkyl;
The tribenzylamine, optionally substituted, in particular on each phenyl group, by a group independently selected from linear or branched C₁ to C₆ alkyl;
The 2,5-dibenzylphenol, optionally substituted, in particular on each carbon atom alpha to the phenyl groups, by one or two groups chosen independently from the linear or branched C₁ to C₆ alkyls, the substituted 2,5-dibenzylphenol being in particular 2,5-bis(2-phenylpropan-2-yl)phenol;
the composition not consisting of biphenyl and naphthalene.

Surprisingly, it has been demonstrated by the inventors that these compounds have a melting point >60° C. individually, but once in a composition according to the aspects of the disclosure, have a lower melting point, or even liquid at room temperature, within a purely aromatic or even polyaromatic eutectic.

And some compounds are proving to be excellent solvents, particularly for molecules containing aromatic residues. For example, the compounds of the disclosure are more effective than toluene in liquid-liquid separation involving these molecules.

According to one embodiment, the composition according to the disclosure is eutectic.

According to one embodiment, the at least two compounds are in particular chosen from the following compounds:

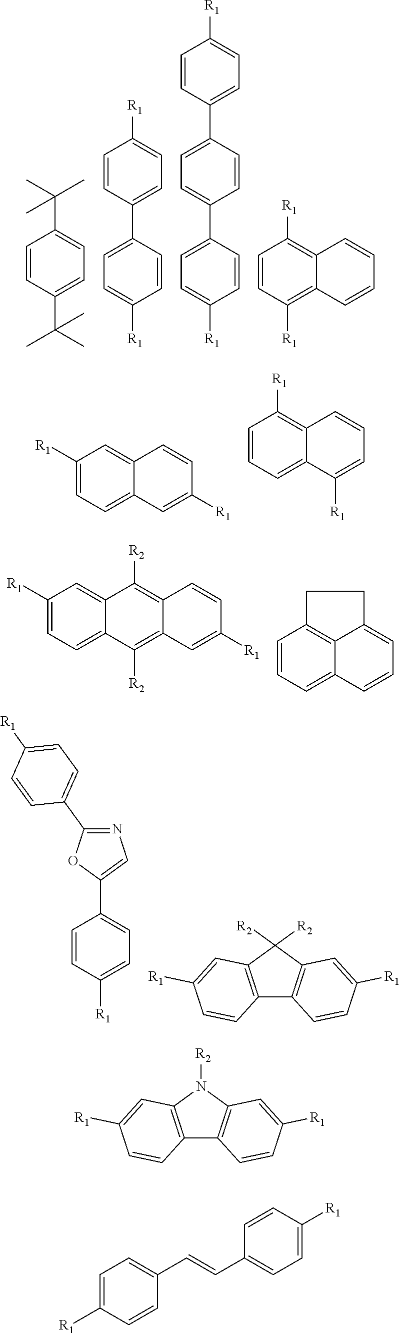

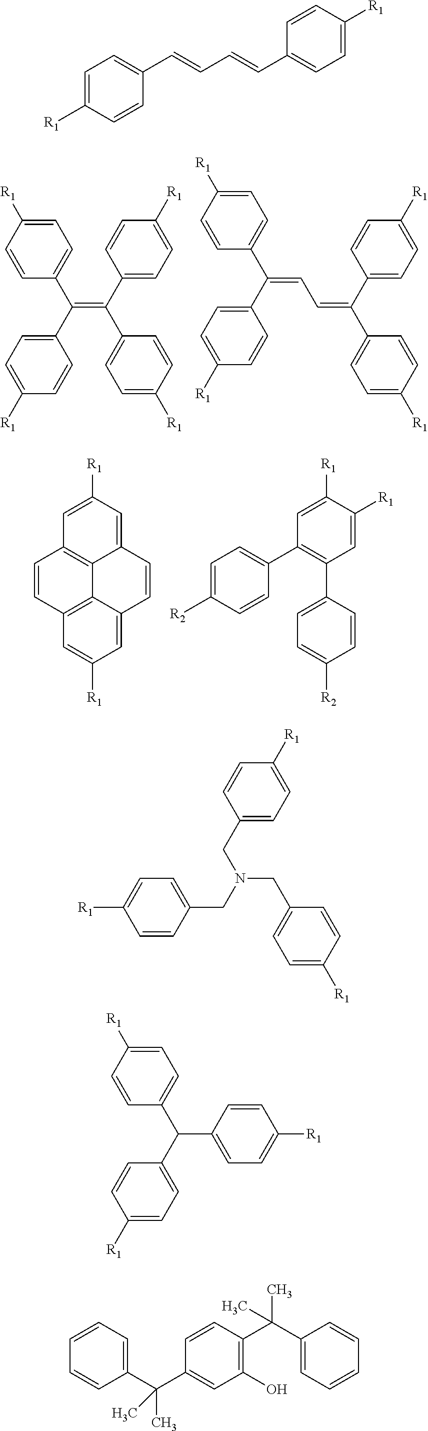

in which R₁ and R₂ are chosen independently of each other,
R₁ being selected from H and linear or branched C₁ to C₆ alkyls, and R₂ being selected from H and the linear or branched C₁ to C₆ alkyls.

According to one embodiment, the disclosure also relates to a composition consisting of; consisting essentially of, or comprising at least two compounds chosen from:
Di-tert-butyl-benzenes;
Biphenyls and terphenyls, optionally substituted by two groups chosen independently from linear or branched C₁ to C₆ alkyls, the terphenyls being in particular ortho-terphenyls, meta-terphenyls or para-terphenyls, in particular para-terphenyls or ortho-terphenyls;
Polycyclic aromatic hydrocarbons selected from naphthalenes, acenaphthenes, anthracenes, which are optionally substituted by two groups independently selected from linear or branched C₁ to C₆ alkyl;
The fluorenes and carbazoles, which are optionally substituted by two or four groups independently selected from linear or branched C₁ to C₆ alkyls;
The 2,5-Diphenyloxazoles, optionally substituted by two groups independently selected from linear or branched C₁ to C₆ alkyls;
The triphenylmethane, optionally substituted, in particular on each phenyl group, by a group selected independently from linear or branched C₁ to C₆ alkyl;
The tribenzylamine, optionally substituted, in particular on each phenyl group, by a group independently selected from linear or branched C₁ to C₆ alkyl;
The 2,5-dibenzylphenol, optionally substituted, in particular on each carbon atom alpha to the phenyl groups, by one or two groups chosen independently from the linear or branched C₁ to C₆ alkyls, the substituted 2,5-dibenzylphenol being in particular 2,5-bis(2-phenylpropan-2-yl)phenol;
the composition not consisting of biphenyl and naphthalene.

According to one embodiment, the at least two compounds are in particular chosen from the following compounds:

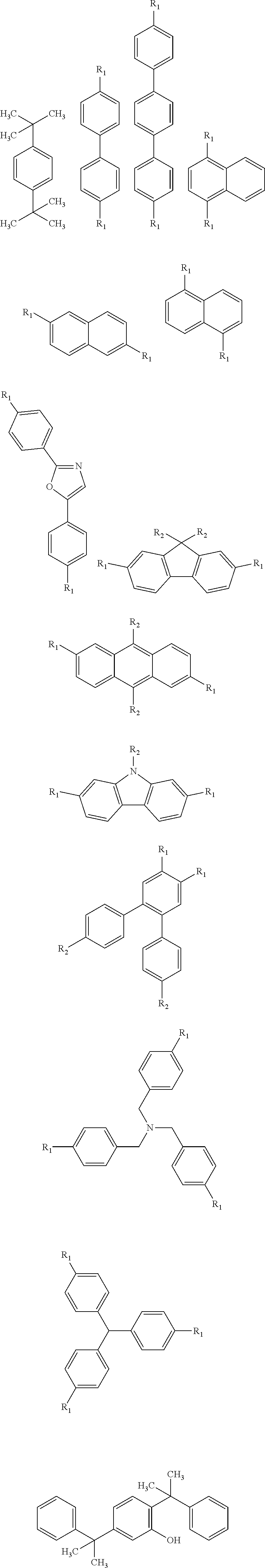

According to one embodiment, the composition of the disclosure is binary, tertiary, quaternary, quinary and so on, depending on the number of components.

According to one embodiment, the composition of the disclosure is binary, tertiary, quaternary or quinary.

According to one embodiment, the composition of the disclosure is:
binary, and the two compounds of the composition are present therein in an amount of 20 to 80 mol %, in particular in an amount of 40 to 60 mol %;
tertiary, and the three compounds of the composition are present therein in an amount of 10 to 55 mol %, in particular in an amount of 25 to 40 mol %;
quaternary, and the four compounds of the composition are present therein in an amount of 5 to 45 mol %, in particular in an amount of 22 to 40 mol % for three of them, and in an amount of 6 to 34 mol % for the last one; or
quinary, and the five compounds of the composition are present therein in an amount of 5 to 45 mol %.

According to another aspect, the disclosure also relates to the use of a composition as solvent, the composition consisting of, consisting essentially of, or comprising at least two compounds chosen from:
Di-tert-butyl-benzenes;
Biphenyls and terphenyls, optionally substituted by two groups chosen independently from linear or branched C₁ to C₆ alkyls, the terphenyls being in particular ortho-terphenyls, meta-terphenyls or para-terphenyls, in particular para-terphenyls or ortho-terphenyls;
Polycyclic aromatic hydrocarbons selected from naphthalenes, acenaphthenes, anthracenes and pyrenes, which are optionally substituted by two groups independently selected from linear or branched C₁ to C₆ alkyls;
The fluorenes and carbazoles, which are optionally substituted by at least two groups independently selected from linear or branched C₁ to C₆ alkyls;
The 2,5-Diphenyloxazoles, optionally substituted by two groups independently selected from linear or branched C₁ to C₆ alkyls;
The compounds of formula (I):

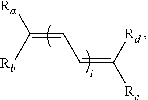

wherein:
i is equal to 0 or 1;
Rₐ, R_{b}, R_{c} and R_{d} are independently selected from H and phenyl, at least two of Rₐ, R_{b}, R_{c} and R_{d} being phenyl;
the compounds of formula (I) being optionally substituted, in particular on the phenyl groups, by two groups chosen independently from the linear or branched C₁ to C₆ alkyls;
The triphenylmethane, optionally substituted, in particular on each phenyl group, by a group selected independently from linear or branched C₁ to C₆ alkyl;
The tribenzylamine, optionally substituted, in particular on each phenyl group, by a group independently selected from linear or branched C₁ to C₆ alkyl;
The 2,5-dibenzylphenol, optionally substituted, in particular on each carbon atom alpha to the phenyl groups, by one or two groups chosen independently from linear or branched C₁ to C₆ alkyls, the substituted 2,5-dibenzylphenol being in particular 2,5-bis(2-phenylpropan-2-yl)phenol.

Surprisingly, it has been demonstrated by the inventors that these compounds have a melting point >60° C. individually, but once in a composition according to the disclosure, have a lower melting point, or even liquid at room temperature, within a purely aromatic or even polyaromatic eutectic.

These purely aromatic or even polyaromatic eutectic compositions are proving to be excellent solvents with unique physicochemical characteristics, particularly for molecules containing aromatic residues. For example, the compounds of the disclosure are more effective than toluene in liquid-liquid separation involving these molecules.

According to one embodiment, the composition is devoid of a further solvent, the further solvent being different from any of the previously defined compounds and mixtures thereof.

According to one embodiment, the use of the disclosure is a use of the composition as previously defined for liquid-liquid separation, liquid-liquid-liquid separation, solid-liquid separation, or solid-liquid-liquid separation.

According to one embodiment, the use of the disclosure is a use of the composition as defined above for the liquid-liquid separation, one of the liquid phases being an aqueous phase, the other liquid phase comprising or consisting of the composition.

According to one embodiment, the use of the disclosure is a use of the composition as defined above for extracting a compound, the compound being in particular chosen from organic compounds and metals, the organic compounds being in particular aromatic or heteroaromatic compounds, or comprising aromatic or heteroaromatic groups,

According to one embodiment, the organic compounds, in particular aromatic or heteroaromatic compounds, or compounds comprising aromatic or heteroaromatic groups, are chosen from:
The plant protective compounds,
The organic pollutants, particularly persistent ones such as dioxins,
The microplastics,
The medicines,
The dyes, and
The carbon black.

### DETAILED DESCRIPTION

### Definitions

As understood here, the value ranges in the form of “x-y” or “from x to y” or “between x and y” include the x and y bounds as well as the integers between these bounds, and the positive real numbers between these bounds and/or integers. For example, “1-5”, or “from “1 to 5” or “between 1 and 5” refer to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

As used herein, the term “alkyl” designates a linear or branched chain alkyl group having the number of carbon atoms indicated before the term, in particular 1 to 4 carbon atoms, such as methyl, ethyl, propyl, isopropyl, butyl, isobutyl, sec-butyl, tert-butyl, etc. Thus, an expression such as “C₁ to C₄ alkyl” designates an alkyl radical containing from 1 to 4 carbon atoms.

By “the composition does not consist of biphenyl and naphthalene” is meant in particular that the composition is not a mixture consisting of unsubstituted biphenyl and unsubstituted naphthalene. In particular, the composition not consisting of biphenyl, optionally substituted with two groups independently selected from linear or branched C₁ to C₆ alkyl, and naphthalene, optionally substituted with two groups independently selected from linear or branched C₁ to C₆ alkyl.

The compositions of the disclosure are eutectics.

By “eutectic” we mean in particular a mixture of two or more substances which has a lower melting temperature than that of each of the individual substances.

The disclosure relates in particular to a composition consisting of, consisting essentially of, or comprising at least two compounds chosen from:
Biphenyls and terphenyls, optionally substituted by two groups chosen independently from linear or branched C₁ to C₆ alkyls, the terphenyls being in particular ortho-terphenyls, meta-terphenyls or para-terphenyls, in particular para-terphenyls or ortho-terphenyls;
Polycyclic aromatic hydrocarbons selected from naphthalenes, acenaphthenes, anthracenes and pyrenes, which are optionally substituted by two groups independently selected from linear or branched C₁ to C₆ alkyls;
The fluorenes and carbazoles, which are optionally substituted by two or four groups independently selected from linear or branched C₁ to C₆ alkyls;
The 2,5-Diphenyloxazoles, optionally substituted by two groups independently selected from linear or branched C₁ to C₆ alkyls;
The compounds of formula (I):

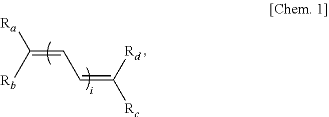

wherein:
i is equal to 0 or 1;
Rₐ, R_{b}, R_{c} and R_{d} are independently selected from H and phenyl, at least two of Rₐ, R_{b}, R_{c} and R_{d} being phenyl;
the compounds of formula (I) being optionally substituted, in particular on the phenyl groups, by two groups chosen independently from linear or branched C₁ to C₆ alkyls;
The triphenylmethane, optionally substituted, in particular on each phenyl group, by a group selected independently from linear or branched C₁ to C₆ alkyl;
The tribenzylamine, optionally substituted, in particular on each phenyl group, by a group independently selected from linear or branched C₁ to C₆ alkyl;
The 2,5-dibenzylphenol, optionally substituted, in particular on each carbon atom alpha to the phenyl groups, by one or two groups chosen independently from the linear or branched C₁ to C₆ alkyls, the substituted 2,5-dibenzylphenol being in particular 2,5-bis(2-phenylpropan-2-yl)phenol;
the composition not consisting of biphenyl and naphthalene.

The disclosure relates in particular to a composition consisting of, consisting essentially of, or comprising at least two compounds chosen from:

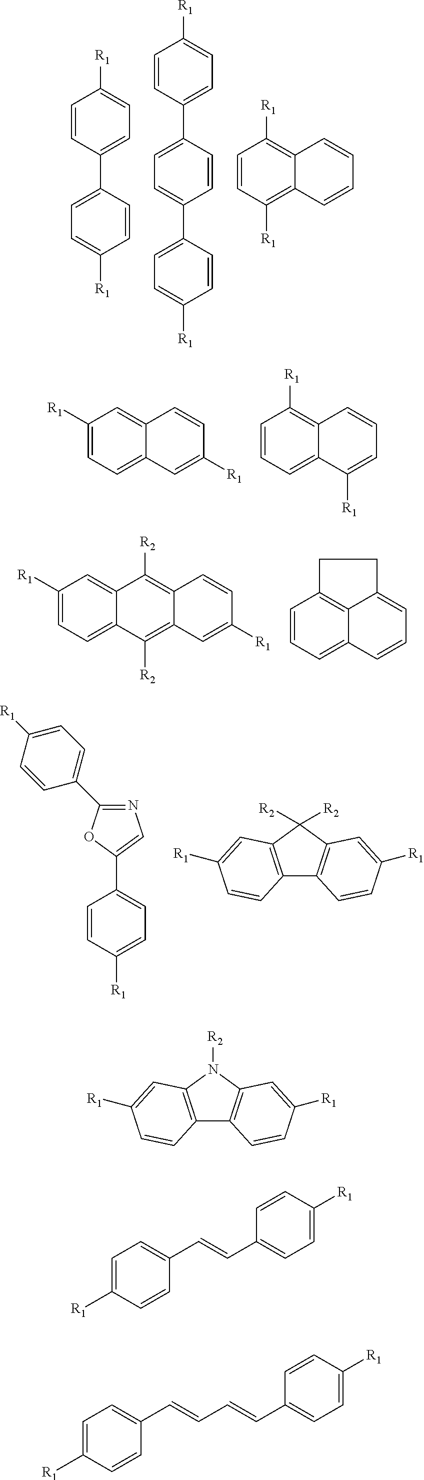

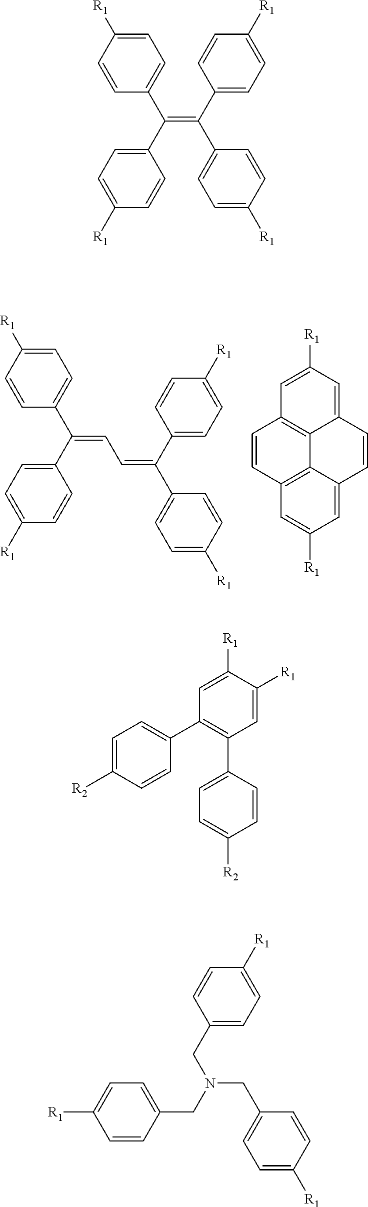

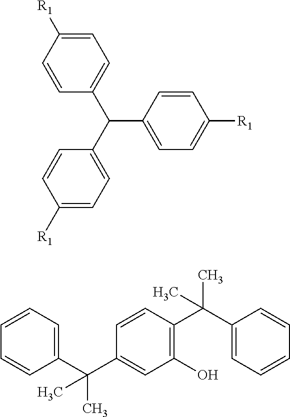

in which R₁ and R₂ are chosen independently of each other,
R₁ being selected from H and linear or branched C₁ to C₆ alkyls, and R₂ being selected from H and the linear or branched C₁ to C₆ alkyls.

The di-tert-butyl-benzene is in particular 1,4-di-tert-butyl-benzene.

The biphenyls and terphenyls include biphenyl, para-terphenyl and meta-terphenyl. The optionally substituted biphenyls are in particular of the following formula:

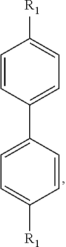

wherein R₁ is selected from H and the linear or branched C₁ to C₆ alkyls.

The optionally substituted terphenyls are in particular of the following formula:

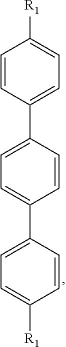

wherein R₁ is selected from H and the linear or branched C₁ to C₆ alkyls,
or the following formula:

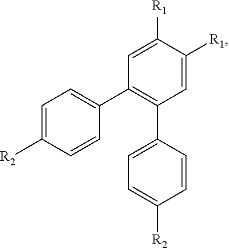

in which R₁ and R₂ are chosen independently of each other,
R₁ being selected from H and linear or branched C₁ to C₆ alkyls, and R₂ being selected from H and the linear or branched C₁ to C₆ alkyls.

The naphthalenes are optionally of one of the following formulae:

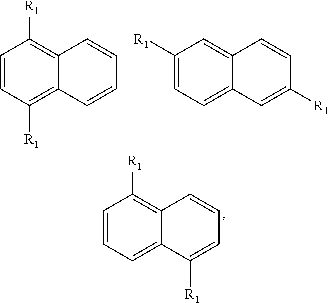

wherein R₁ is selected from H and the linear or branched C₁ to C₆ alkyls.

The acenaphthene is in particular unsubstituted.

The optionally substituted anthracenes are in particular of the following formula:

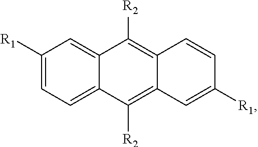

wherein R₁ is selected from H and the linear or branched C₁ to C₆ alkyl, and R₂ is selected from H and the linear or branched C₁ to C₆ alkyls.

The optional pyrenes are in particular of the following formula:

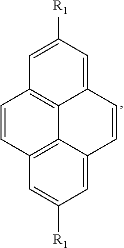

wherein R₁ is selected from H and the linear or branched C₁ to C₆ alkyls.

The 2,5-diphenyloxazoles optionally are in particular of the following formula:

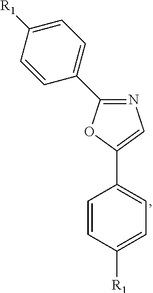

wherein R₁ is selected from H and the linear or branched C₁ to C₆ alkyls.

The optionally substituted fluorenes are in particular of the following formula:

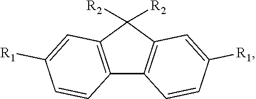

wherein R₁ is selected from H and the linear or branched C₁ to C₆ alkyl, and R₂ is selected from H and the linear or branched C₁ to C₆ alkyls.

The optionally substituted carbazoles are in particular of the following formula:

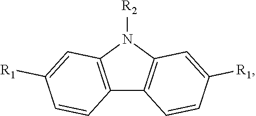

wherein R₁ is selected from H and the linear or branched C₁ to C₆ alkyl, and R₂ is selected from H and the linear or branched C₁ to C₆ alkyls.

The compounds of formula (I) are in particular chosen from 1,2-diphenylethenes, 1,1,2,2-tetraphenylethenes, 1,2-diphenylbutadienes, 1,1,4,4-tetraphenylbutadienes, optionally substituted, in particular on the phenyl groups, by two groups chosen independently from the linear or branched C₁ to C₆ alkyls.

The optionally substituted 1,2-tetraphenyl ethene compounds are in particular of the following formula:

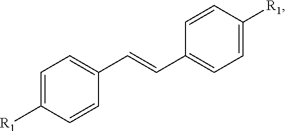

wherein R₁ is selected from H and the linear or branched C₁ to C₆ alkyls.

The optionally substituted 1,1,2,2-tetraphenylethenes are in particular of the following formula:

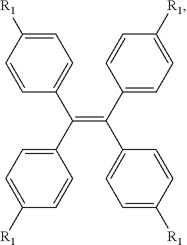

wherein R₁ is selected from H and the linear or branched C₁ to C₆ alkyls.

The optionally substituted 1,2-diphenylbutadienes are in particular of the following formula:

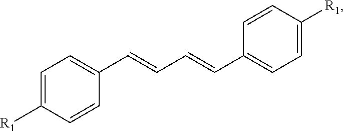

wherein R₁ is selected from H and the linear or branched C₁ to C₆ alkyls.

The optionally substituted 1,1,4,4-tetraphenylbutadienes are in particular of the following formula:

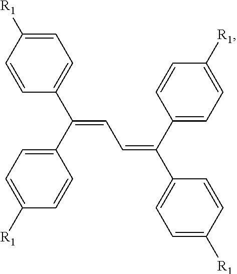

wherein R₁ is selected from H and the linear or branched C₁ to C₆ alkyls.

The triphenylmethane, optionally substituted is in particular of the following formula:

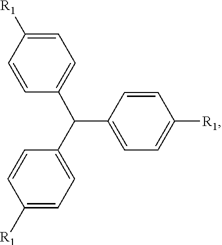

wherein R₁ is selected from H and the linear or branched C₁ to C₆ alkyls.

The tribenzylamine, optionally substituted, is in particular of the following formula:

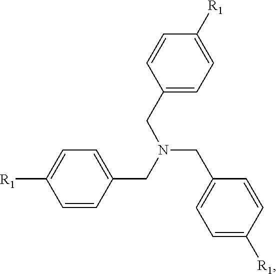

wherein R₁ is selected from H and the linear or branched C₁ to C₆ alkyls.

The 2,5-dibenzylphenol is in particular the 2,5-bis(2-phenylpropan-2-yl)phenol of the following formula:

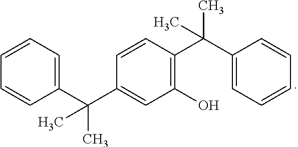 [SMILES: CC(C)(c1ccccc1)c1ccc(C(C)(C)c2ccccc2)c(O)c1]

The composition according to the disclosure can be a composition wherein the at least two compounds are of the same formula as defined above, differing only in the meaning of the R₁ and/or R₂ group.

According to a particular embodiment, the composition according to the disclosure consists of at least two compounds as defined above.

The composition, in particular the eutectic, according to the disclosure can be binary (two-component), tertiary (three-component), quaternary (four-component), quinary (five-component) and so on.

According to one embodiment, the composition of the disclosure is tertiary, quaternary or quinary. This is likely to enable compositions with a lower melting temperature, for example much lower than ambient temperature, to be obtained if desired.

According to a particular embodiment, the composition consists of, consists essentially of, or comprises:
a biphenyl, optionally substituted by two groups independently selected from linear or branched C₁ to C₆ alkyls, in particular biphenyl; and
a 2,5-diphenyloxazole, optionally substituted with two groups independently selected from linear or branched C₁ to C₆ alkyls, in particular 2,5-diphenyloxazole.

According to a particular embodiment, the composition consists of, consists essentially of, or comprises:
a biphenyl, optionally substituted by two groups independently selected from the linear or branched C₁ to C₆ alkyl, in particular biphenyl;
a 2,5-diphenyloxazole, optionally substituted by two groups independently selected from linear or branched C₁ to C₆ alkyl, in particular the 2,5-diphenyloxazole; and
a carbazole, optionally substituted with two groups independently selected from the linear or branched C₁ to C₆ alkyls, in particular 9-ethylcarbazole.

According to a particular embodiment, the composition consists of, consists essentially of, or comprises:
a biphenyl, optionally substituted by two groups independently selected from the linear or branched C₁ to C₆ alkyl, in particular biphenyl;
a 2,5-diphenyloxazole, optionally substituted by two groups independently selected from the linear or branched C₁ to C₆ alkyls, in particular the 2,5-diphenyloxazole;
a carbazole, optionally substituted by two groups independently selected from the linear or branched C₁ to C₆ alkyls, in particular the 9-ethylcarbazole; and
a diterbutylbenzene, in particular 1,4-Diterbutylbenzene, a second biphenyl, optionally substituted with two groups independently selected from C₁ to C₆ different from the first, in particular diterbutylbiphenyl, and/or a fluorene, in particular diterbutylfluorene.

According to a particular embodiment, the composition consists of, consists essentially of, or comprises: triphenylmethane,
o-terphenyl,
tribenzylamine, and
2,4-bis(alpha,alpha-dimethylbenyl)phenol.

According to another aspect, the disclosure also relates to the use of a composition as described above as a solvent.

All the embodiments previously defined in relation to the composition of the disclosure also apply here, alone or in combination.

According to a particular embodiment, the composition does not consist of biphenyl and naphthalene. In fact, their binary eutectic may not have a low enough melting point depending on the intended use.

These compositions, as solvents, can be used in all extraction and/or separation techniques well known to those skilled in the art involving an organic solvent.

### EXAMPLES

### Example 1: Preparation of Compositions According to the Disclosure

The binary mixtures B_{A}, ternary mixtures T_{A}, and quaternary mixtures Q_{A} Q_{B} and Q_{C} were prepared and characterized as follows.

The melting temperature of the mixtures was determined by Differential Scanning Calorimetry (DSC).

| Molecule | Molar proportion | Tₘₑₗₜ (° C.) |
|---|---|---|
| Biphenyl | 44% | 69 |
| Diphenyleoxazole (PPO) | 56% | 70 |
| Eutectic | | T_{eutectic} (° C.) |
| B_{A} | | 39 |

| Molecule | Molar proportion | Tₘₑₗₜ |
|---|---|---|
| Biphenyl | 35.0% | 69 |
| 9-ethylcarbazole | 37.5% | 70 |
| Diphenyleoxazole (PPO) | 27.5% | 70 |
| Eutectic | | T_{eutectic} (° C.) |
| T_{A} | | 19 |

| Molecule | Molar proportion | Tₘₑₗₜ |
|---|---|---|
| Biphenyl | 31.6% | 69 |
| 9-ethylcarbazole | 33.7% | 70 |
| Diphenyleoxazole (PPO) | 24.8% | 70 |
| Diterbutylbenzene (DTBB) | 9.9% | 77 |
| Eutectic | | T_{eutectic} (° C.) |
| Q_{A} | | 16 |

| Molecule | Molar proportion | Tₘₑₗₜ |
|---|---|---|
| Biphenyl | 32.5% | 69 |
| 9-ethylcarbazole | 34.7% | 70 |
| Diphenyleoxazole (PPO) | 25.6% | 70 |
| Diterbutybiphenyl (DTBP) | 7.3% | 127 |
| Eutectic | | T_{eutectic} (° C.) |
| Q_{B} | | 16 |

| Molecule | Molar proportion | Tₘₑₗₜ |
|---|---|---|
| Biphenyl | 32.6% | 69 |
| 9-ethylcarbazole | 34.8% | 70 |
| Diphenyleoxazole (PPO) | 25.6% | 70 |
| Diterbutylfluorene (DTBF) | 7.0% | 123 |
| Eutectic | | T_{eutectic} (° C.) |
| Q_{C} | | 15 |

### Example 2: Use of Compositions According to the Disclosure as Solvent for Liquid-Liquid Separation

The mixture Q_{A} of Example 1 was evaluated in the extraction of a dye in the aqueous phase. The colourant is coumarin 151, for example.

To do this, the coumarin 151 was dissolved in the water at pH 10. The solution thus obtained is fluorescent (in green).

When this aqueous phase was extracted with an equal quantity of toluene, only partial extraction of the dye by the toluene was visualized (green fluorescence remaining in the aqueous phase).

However, when the same aqueous phase was extracted with the mixture Q_{A}, no residual green fluorescence was observed in the extracted aqueous phase, indicating that the dye had been completely extracted from the aqueous medium.

### Example 3: Preparation of Compositions According to the Disclosure and Evaluation of Extraction Using the Compositions

Quaternary mixtures comprising the following compounds have been prepared and characterized:
triphenylmethane,
o-terphenyl,
n,n-dibenzylaniline, and
2,4-bis(alpha,alpha-dimethylbenyl)phenol.
And in particular:
16.3 mol % triphenylmethane,
40.6 mol % o-terphenyl,
21.1 mol % tribenzylamine,
22.8 mol % 2,4-bis(alpha,alpha-dimethylbenyl)phenol.

The melting temperature of these compounds was determined by DSC, and the melting temperature of the mixture calculated using the Schradler-van Larr equation.
triphenylmethane: 93.6° C. and 19.6 kJ/mol,
o-terphenyl: 56.9° C. and 16.1 kJ/mol,
tribenzylamine: 69.3° C. and 22.4 kJ/mol,
2,4-bis(alpha,alpha-dimethylbenyl)phenol: 64.9° C. and 22.9 kJ/mol.

This gives a melting point for the eutectic of 13° C.

Extraction tests using this composition were carried out on 5 compounds. Extraction was assessed by UV-visible spectroscopy.

The partition coefficient of the molecule to be extracted between the organic (hydrophobic eutectic solvent) and aqueous phases K_{org/aq} was calculated using the following equation:

$${K}_{org / aq} = ( ( [ {C}_{aq , i} ] - [ {C}_{aq , f} ] ) * {Vol}_{aq} ) / ( [ {C}_{aq , f} ] * {Vol}_{org} ) ,$$

where:
[C_{aq,i}] and [C_{aq,f}] are the concentrations before and after extraction in the aqueous phase, and
Vol_{aq} and VOl_{org} are the volumes of the aqueous and organic phases.

The molecules to be extracted and the corresponding partition coefficient calculated are shown below:
Bisphenol-A (polyaromatic pollutant used as an additive in the polymers, banned for certain applications because of its toxicity): K=10;
Nicotine (an aromatic carcinogen): K=45;
Lidocaine (medicinal, aromatic): K>40;
2-naphthol (polyaromatic): K=50;
Rhodamine B (dye, polyaromatic): K=65.

In the detailed description herein, references to “one embodiment”, “an embodiment”, “an example embodiment”, “one or more embodiments”, “some embodiments”, etc., indicate that the embodiment or embodiments described may include a particular feature, structure, or characteristic, but every embodiment may not necessarily include the particular feature, structure, or characteristic. Moreover, such phrases are not necessarily referring to the same embodiment or embodiments. In addition, when a particular feature, structure, or characteristic is described in connection with an embodiment or embodiments, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments. Thus, it will be appreciated that embodiments of the present disclosure may employ any combination of features described herein. All such combinations or sub-combinations of features are within the scope of the present disclosure.

Throughout this specification, terms of art may be used. These terms are to take on their ordinary meaning in the art from which they come, unless specifically defined herein or the context of their use would clearly suggest otherwise.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

The embodiments of the invention in which an exclusive property or privilege is claimed are defined as follows:

1. A composition comprising two or more compounds chosen from:
Di-tert-butyl-benzenes;
Biphenyls and terphenyls, optionally substituted by two groups chosen independently from linear or branched C₁ to C₆ alkyls, the terphenyls being ortho-terphenyls, meta-terphenyls or para-terphenyls;
Polycyclic aromatic hydrocarbons selected from naphthalenes, acenaphthenes, anthracenes and pyrenes, the polycyclic aromatic hydrocarbons being optionally substituted by two groups independently selected from linear or branched C₁ to C₆ alkyls;
Fluorenes and carbazoles, which are optionally substituted by two or four groups independently selected from linear or branched C₁ to C₆ alkyls;
2,5-Diphenyloxazoles, optionally substituted by two groups independently selected from linear or branched C₁ to C₆ alkyls;
Compounds of formula (I): 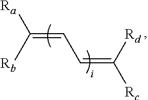
wherein:
i is equal to 0 or 1;
Rₐ, R_{b}, R_{c} and R_{d} are independently selected from H and phenyl, at least two of Rₐ, R_{b}, R_{c} and R_{d} being phenyl;
the compounds of formula (I) being optionally substituted by two groups chosen independently from linear or branched C₁ to C₆ alkyls;
Triphenylmethane, optionally substituted by a group selected independently from linear or branched C₁ to C₆ alkyl;
Tribenzylamine, optionally substituted by a group independently selected from linear or branched C₁ to C₆ alkyl; and
2,5-dibenzylphenol, optionally substituted by one or two groups chosen independently from the linear or branched C₁ to C₆ alkyls;
the composition not consisting of biphenyl and naphthalene.

2. The composition according to claim 1, which is eutectic.

3. The composition according to claim 1, wherein the at least two compounds are chosen from the following compounds: 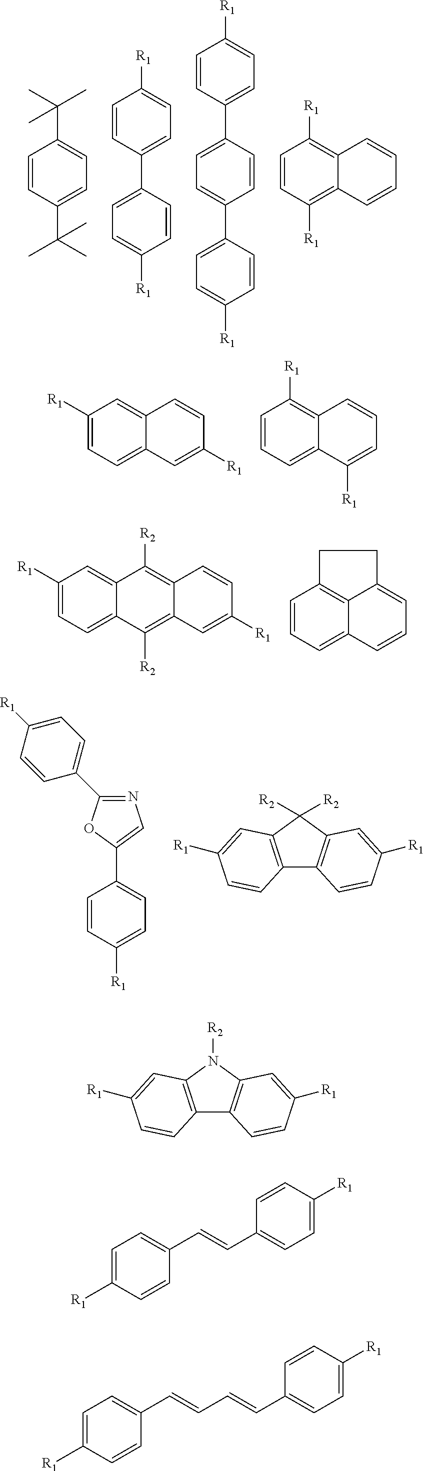 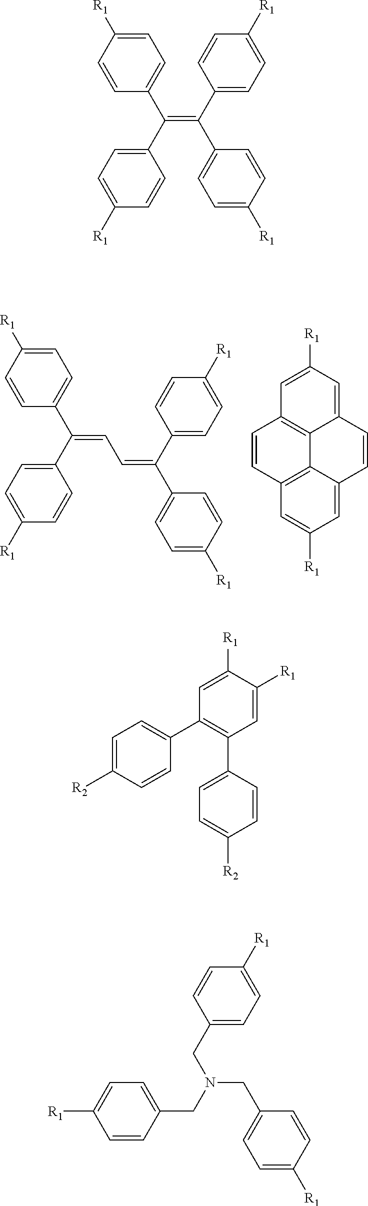 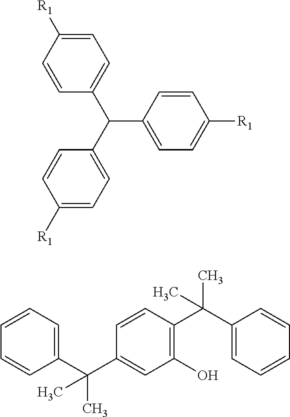
wherein R₁ and R₂ are chosen independently of each other,
R₁ being selected from H and linear or branched C₁ to C₆ alkyls, and R₂ being selected from H and linear or branched C₁ to C₆ alkyls.

4. The composition according to claim 1, which is binary, tertiary, quaternary or quinary.

5. The composition according to claim 1, which is:
binary, and the two compounds of the composition are present therein in an amount of 20 to 80 mol %;
tertiary, and the three compounds of the composition are present therein in an amount of 10 to 55 mol %;
quaternary, and the four compounds of the composition are present therein in an amount of 5 to 45 mol %; or
quinary, and the five compounds of the composition are present therein in an amount of 5 to 45 mol %.

6. A method of use of a composition as solvent, the composition comprising at least two compounds chosen from:
Di-tert-butyl-benzenes;
Biphenyls and terphenyls, optionally substituted by two groups chosen independently from linear or branched C₁ to C₆ alkyls, the terphenyls being ortho-terphenyls, meta-terphenyls or para-terphenyls;
Polycyclic aromatic hydrocarbons selected from naphthalenes, acenaphthenes, anthracenes and pyrenes, which are optionally substituted by two groups independently selected from linear or branched C₁ to C₆ alkyls;
Fluorenes and carbazoles, which are optionally substituted by at least two groups independently selected from linear or branched C₁ to C₆ alkyls;
2,5-Diphenyloxazoles, optionally substituted by two groups independently selected from linear or branched C₁ to C₆ alkyls;
Compounds of formula (I): 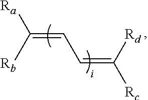
wherein:
i is equal to 0 or 1;
Rₐ, R_{b}, R_{c} and R_{d} are independently selected from H and phenyl, at least two of Rₐ, R_{b}, R_{c} and R_{d} being phenyl;
the compounds of formula (I) is optionally substituted by two groups chosen independently from the linear or branched C₁ to C₆ alkyls;
Triphenylmethane, optionally substituted by a group selected independently from linear or branched C₁ to C₆ alkyl;
Tribenzylamine, optionally substituted by a group independently selected from linear or branched C₁ to C₆ alkyl; and
2,5-dibenzylphenol, optionally substituted by one or two groups chosen independently from the linear or branched C₁ to C₆ alkyls.

7. The method of use according to claim 6, wherein the composition is devoid of a further solvent, the further solvent being other than any of the compounds defined in claim 6 and mixtures thereof.

8. The method of use according to claim 6, wherein the use as a solvent is for liquid-liquid separation, liquid-liquid-liquid separation, solid-liquid separation, or solid-liquid-liquid separation.

9. The method of use according to claim 6, wherein the use as a solvent is for liquid-liquid separation, one of the liquid phases being an aqueous phase, the other liquid phase comprising or consisting of the composition.

10. The method of use according to claim 6, wherein the use as a solvent is for extracting a compound, the compound being chosen in particular from the organic compounds and metals,
the organic compounds are chosen from:
a plant protective compound,
an organic pollutant,
a microplastic,
a medicine,
a dyes, and
a carbon black.

11. The composition of claim 1, wherein the terphenyls are para-terphenyls or ortho-terphenyls.

12. The composition of claim 1, wherein the substituted 2,5-dibenzylphenol is 2,5-bis(2-phenylpropan-2-yl)phenol.

13. The composition of claim 1, wherein:
the compounds of formula (I) are optionally substituted on the phenyl group;
the triphenylmethane is optionally substituted on each phenyl group;
the tribenzylamine is optionally substituted on each phenyl group; and
the 2,5-dibenzylphenol is optionally substituted on each carbon atom alpha to the phenyl groups.

14. The composition of claim 5, wherein:
when the composition is binary, the two compounds of the composition are present therein in an amount of 40 to 60%;
when the composition is tertiary, the three compounds of the composition are present therein in an amount of 25 to 40 mol %; and
when the composition is quaternary, the four compounds of the composition are present therein in an amount of 22 to 40 mol % for three of the four compounds, and in an amount of 6 to 34 mol % for the fourth of the four compounds.

15. The method of use of claim 6, wherein the terphenyls are para-terphenyls or ortho-terphenyls.

16. The method of claim 6, wherein the substituted 2,5-dibenzylphenol is 2,5-bis(2-phenylpropan-2-yl)phenol.

17. The method of use of claim 6, wherein:
the compounds of formula (I) are optionally substituted on the phenyl group;
the triphenylmethane is optionally substituted on each phenyl group;
the tribenzylamine is optionally substituted on each phenyl group; and
the 2,5-dibenzylphenol is optionally substituted on each carbon atom alpha to the phenyl groups.

18. The method of use of claim 10, wherein the organic compounds are an aromatic compound or a heteroaromatic compound.

19. The method of use of claim 10, wherein the organic compounds comprise an aromatic substituent group or a heteroaromatic substituent group.

20. The method of claim 10, wherein the organic pollutant is dioxin.
